(19) United States
(12) Patent Application Publication
(10) Pub. No.: US 2016/0074151 A1
(43) Pub. Date: Mar. 17, 2016

(54) SYSTEMS AND METHODS FOR ALIGNMENT AND SITE PREPARATION OF ROTATOR CUFF GRAFTS

(71) Applicants: Ferris M. PFEIFFER, Columbia, MO (US); Matthew J. SMITH, Columbia, MO (US); James L. COOK, Columbia, MO (US); THE CURATORS OF THE UNIVERSITY OF MISSORURI, Columbia, MO (US)

(72) Inventors: Ferris M. Pfeiffer, Columbia, MO (US); Matthew J. Smith, Columbia, MO (US); James L. Cook, Columbia, MO (US)

(73) Assignee: The Curators of the University of Missouri, Columbia, MO (US)

(21) Appl. No.: 14/888,889

(22) PCT Filed: May 5, 2014
(86) PCT No.: PCT/US14/36814

§ 371 Date: Nov. 3, 2015

(51) Int. Cl. A61F 2/08; A61B 17/16; A61B 17/17

(52) U.S. Cl. CPC A61F 2/0805; A61B 17/1714; A61B 17/1633; A61B 17/1684; A61F 2/0811; A61B 2017/1778; A61F 2002/087

(57) ABSTRACT

A graft-positioning system that includes a body further comprising a shaft, a handle engaged to the first end of the shaft, a guide rest engaged to the second end of the shaft and a flange engaged to the guide. In addition, the shaft also includes a plurality of pin channels that extend from the first end to the second end of the shaft. The graft-positioning system may also include plurality of guide pins that are configured and arranged to be received within the plurality of pin channels. Additional embodiments provide an arthroscopic cutter that includes a tubular body and a drive shaft attached to a cutting head at one end and situated within a lumen defined within the tubular body. The tubular body further includes a guide rest engaged to the distal end of the tubular body and a flange engaged to the guide rest.

### CROSS-REFERENCE TO RELATED APPLICATION

This application claims priority to U.S. Provisional Application Ser. No. 61/819,415 filed on May 3, 2013 and entitled “SYSTEMS AND METHODS FOR ALIGNMENT AND SITE PREPARATION OF ROTATOR CUFF GRAFTS” which is hereby incorporated by reference in its entirety.

### FIELD

The present document generally relates to site preparation for the surgical repair of rotator cuff injuries, and in particular to systems and methods for site preparation and alignment of rotator-cuff grafts used in the surgical repair of major rotator cuff tears.

### BACKGROUND

Rotator-cuff tears are relatively common injuries, and a significant portion of rotator-cuff tears may require surgical repair. Massive rotator-cuff tears may account for approximately 30% of these surgically repaired rotator-cuff tears. Existing treatments for large to massive chronic rotator-cuff tears have not yielded consistently good results, with failure rates as high as 94% reported in some studies. Currently, the most commonly used surgical treatment is direct repair of native tendon to bone. However, direct repair treatments are associated with biomechanically inferior repair constructs, exhibiting only 18% and 31% of the strength of the normal rotator cuff at 6 and 12 weeks after repair, respectively. Failure to reestablish a normal bone-tendon junction at the repair site appears to be a prevalent reason for the inability to obtain functional rotator-cuff repairs and for the resultant failures. Recent advances in surgical procedures used for massive rotator cuff repairs make use of bone-tendon allografts in which intact bone-tendon units are implanted via a recipient channel formed in the native bone resulting in enhancements to tissue healing and integration, as well as biomechanical strength.

The method of preparing the native bone surface of the humeral head and the alignment of the allograft and associated fasteners with the prepared bone surface may influence the successful outcome of a major rotator cuff repair. Predominantly, visual procedures are used by medical practitioners during the preparation of the allograft implantation site and the subsequent positioning and alignment of the allograft during implantation. However, these visual procedures may have considerable volatility and may introduce inaccuracies in the alignment of the allograft within the prepared surface.

A need exists for devices and methods for preparation of an allograft implantation site as well as for the positioning and alignment of an allograft that does not require visual procedures.

### SUMMARY

In some embodiments, a graft-positioning system may include a body further including a shaft, a handle, and a guide rest. In particular, the shaft may include a first end and a second end, with the handle engaged to the first end of the shaft and the guide rest engaged to the second end of the shaft. In addition, in some embodiments, the shaft may also include a plurality of pin channels that extend from the first end of the shaft to the second end of the shaft. Moreover, the body may also include a flange that is engaged to the guide rest. The graft-positioning system may also include a plurality of guide pins that are configured and arranged to be received within the plurality of pin channels.

In another embodiment, a method is provided for preparing a bone to receive a graft. For example, the method may include providing a graft-positioning system that includes a body and at least one guide pin. In one embodiment, the body may include at least one pin channel that is configured and arranged to receive at least a portion of the guide pin. The body may also include a guide rest that is capable of engaging a flange. Moreover, the guide pin may also include an engagement end and an opposing receiving end. In some embodiments, the method may further include engaging the bone with the body such that the flange contacts a portion of the bone. The method may also include positioning the guide pin through the pin channel such that the engagement end of the guide pin contacts the bone. Thereafter, the method also includes actuating the guide pin to engage the guide pin and the bone and then removing the body such that the guide pin remains engaged to the bone.

In another embodiment, an arthroscopic cutter is provided that includes a tubular body, a guide rest being engaged to the distal end of the tubular body, and a flange being engaged to the guide rest, as well as a lumen extending from a proximal end to a distal end of the tubular body. The arthroscopic cutter also includes a drive shaft attached to a cutting head at one end and situated within the lumen. A second end of the drive shaft opposite to the cutting head protrudes from the proximal end of the tubular body. The cutting head is stowed within the lumen near the distal end of the tubular body in a stowed position. The cutting head protrudes slightly from the distal end of the lumen of the tubular body in an activated position. The body and the guide rest may be integral with respect to each other. The flange may be movable with respect to the guide rest. The guide rest may include a guide channel. The flange may be immovable with respect to the guide rest. The second end of the drive shaft may terminate in an attachment fitting for attaching a power driver. The arthroscopic cutter may also include a retraction spring wrapped circumferentially around the drive shaft between the cutting head and the second end. The retraction spring may be attached to the drive shaft at a first end and the retraction spring may be attached to a lumen wall within the body at a second end opposite to the first end. The retraction spring may generate essentially no force when the cutting head is in the stowed position and the retraction spring may generate a retracting force when the cutting head is in the activated position.

In another embodiment, a shell allograft is provided that includes a bone plate with a contact surface and an exposed surface separated by a thickness, as well as a tendon joined to the bone plate in an intact bone-tendon junction. The contact surface is contoured to conform to an exposed surface of a bone, and the bone plate may define at least one fastener opening extending through the thickness of the bone plate. The thickness of the bone plate may range from about 2 mm to about 6 mm. The height of the bone plate may range from about 10 mm to about 25 mm. The width of the bone plate may range from about 10 mm to about 50 mm.

Additional objectives, advantages and novel features will be set forth in the description which follows or will become apparent to those skilled in the art upon examination of the drawings and detailed description which follows.

### BRIEF DESCRIPTION OF THE DRAWINGS

FIG. 1 is an isometric view of a graft-positioning system;

FIG. 2 is a side view of an embodiment of the graft-positioning system;

FIG. 3 is a simplified illustration of the graft-positioning system of FIG. 1 engaged to a bone;

FIG. 4 is a side view of the graft-positioning system of FIG. 1 with phantom views of a guide channel and pin channels;

FIG. 5 is a rear perspective view of the graft-positioning system of FIG. 4;

FIG. 6 is an isometric view of an embodiment of the graft-positioning system;

FIG. 7 is a side view of the graft-positioning system of FIG. 6 with a phantom view of pin channels;

FIG. 8 is a rear perspective view of the graft-positioning system of FIG. 7;

FIG. 9 is a simplified illustration of the graft-positioning system of FIG. 1 engaged to a bone;

FIG. 10 is a simplified illustration of the graft-positioning system of FIG. 1 engaged to a bone with guide pins engaged to a bone;

FIG. 11 is a simplified illustration of a plurality of guide pins engaged to a bone;

FIG. 12 is a side view of an arthroscopic cutting tool with phantom views of a driveshaft and a retraction spring;

FIG. 13 is a front perspective view of an arthroscopic cutting tool with phantom views of a driveshaft and a retraction spring;

FIG. 14 is a front perspective view of an arthroscopic cutting tool engaged to a bone;

FIG. 15 is a front view of a shell allograft attached to a humerus head; and

FIG. 16 is a lateral view of a shell allograft attached to a humerus head.

Corresponding reference characters indicate corresponding elements among the view of the drawings. The headings used in the figures should not be interpreted to limit the scope of the claims.

### DETAILED DESCRIPTION

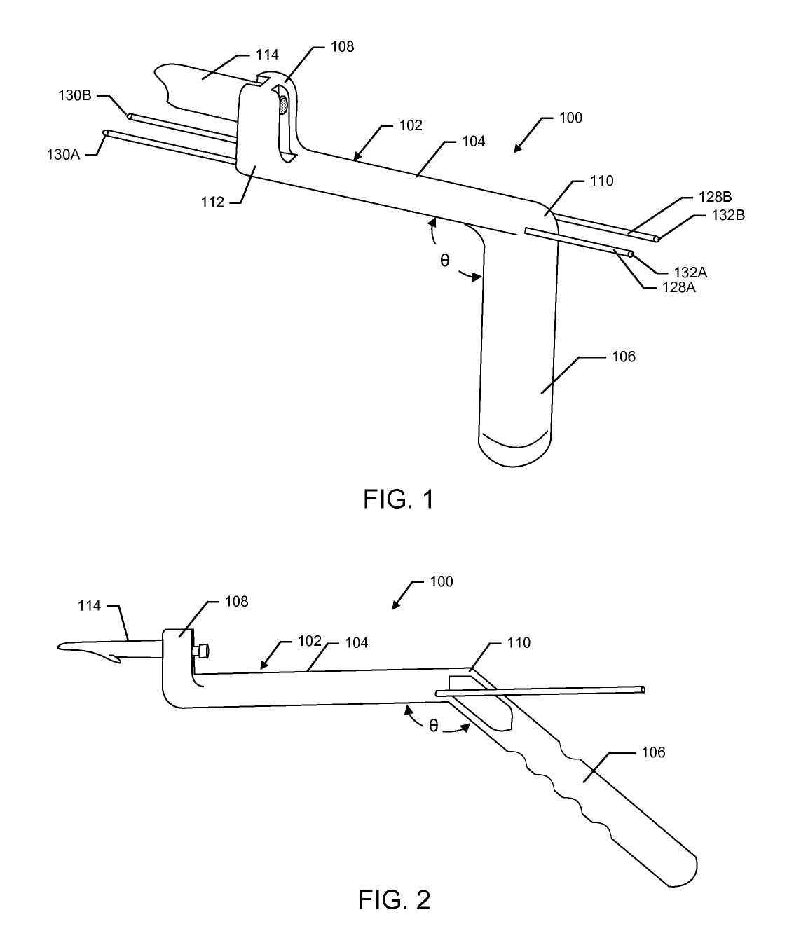

Referring to the drawings, various aspects of a graft-positioning system are illustrated and generally indicated as 100 in FIGS. 1-5 and 9-11. As described in greater detail herein, the graft-positioning system 100 may be employed by healthcare professionals in connection with one or more medical procedures for alignment and site preparation for implantation of various grafts. For example, the graft-positioning system 100 may be employed by a healthcare professional (e.g., an orthopedic surgeon and/or other healthcare professionals working under the guidance of one or more orthopedic surgeons) for preparing a site on one or more patients to receive a graft during an arthroscopic procedure. In some embodiments, the healthcare professional may use the graft-positioning system 100 in preparing a site on a patient to receive a rotator-cuff graft including, but not limited to, an allogenic or autogenic graft. In particular, the graft-positioning system 100 may be used in disposing one or more guide pins within or at least partially through a bone including, but not limited to, a humerus. The guide pins, in one aspect, may be used to guide the placement of surgical tools including, but not limited to, cutters and cannulated drills that may be used to form depressions within the bone. Non-limiting examples of depressions associated with the implantation of a rotator-cuff graft include, but are not limited to: bores, channels, holes, sockets, and any other suitable depression.

Thereafter, one or more grafts may be situated relative to the depressions using the one or more guide pins to orient the one or more grafts appropriately to provide an environment for successful graft implantation. In other embodiments, the graft-positioning system 100 may be used by healthcare professionals or other individuals in preparing a site on a patient to receive any other type of graft or in any other suitable medical procedure. Although the following discussion proceeds in the context of the use of the graft-positioning system 100 for rotator-cuff grafts, this discussion is only exemplary and is not intended the limit the scope of the invention.

As illustrated in FIG. 1, the graft-positioning system 100 is configured for use by healthcare professionals during medical procedures including, but not limited to, arthroscopic surgeries. In some embodiments, the graft-positioning system 100 may include a body 102 having a shaft 104, a handle 106, and a guide rest 108. In particular, in some embodiments the body 102 may be formed such that the shaft 104, the handle 106, and the guide rest 108 are substantially or completely integral with each other. For example, in one embodiment, the body 102 may be manufactured from surgical-grade steel or a substantially similar material such that some or all of the constituents of the body 102 are molded, forged, casted, or otherwise formed as a single integrated element at substantially the same time. In other embodiments, the shaft 104, the handle 106, and/or the guide rest 108 may be made at the same or different times and coupled together at a later time. For example, at least some portions of the shaft 104, the handle 106, and/or the guide rest 108 may be fabricated from an appropriate material (e.g., surgical-grade steel) and coupled together using conventional coupling techniques including, but not limited to, welding, brazing, adhesives, and/or using one or more mechanical coupling devices including, but not limited to, screws, nuts, bolts, clamps, and any other suitable mechanical coupling devices.

In some embodiments, the body 102 may be configured and arranged to enable a healthcare professional to prepare a graft implantation site. Specifically, the shaft 104 may be generally centrally located with respect to the handle 106 and the guide rest 108. For example, the shaft 104 may include a first end 110 and a second end 112, with the handle 106 and the guide rest 108 positioned at the first and second ends 110, 112, respectively. In particular, the first end 110 of the shaft 104 may oppose the second end 112 of the shaft 104. In other words, in some embodiments the handle 106 may be formed such that the first end 110 of the shaft 104 and the handle 106 are substantially integral with each other. In other embodiments, the handle 106 may be coupled to the first end 110 of the shaft 104. Similarly, the guide rest 108 may be formed such that the second end 112 of the shaft 104 and the guide rest 108 are substantially integral with each other. In other embodiments, the guide rest 108 may be coupled to the second end 112 of the shaft 104.

In some embodiments, the handle 106 may be positioned to steady, secure, and/or move the graft-positioning system 100 during use. In various embodiments, the handle angle θ, defined herein as the angle at which the handle 106 extends from the shaft 104 may range from about 90° to about 270°. In one non-limiting example, the handle 106 may extend from the first end 110 of the shaft 104 in a substantially perpendicular manner (i.e. θ=90°), as illustrated in FIG. 1. In another non-limiting example, the handle angle θ may be between about 90° and about 180°, as illustrated in FIG. 2. As a result, the healthcare professional employing the graft-positioning system 100, may control the positioning of the body 102 during the medical procedure by engaging the handle 106.

Moreover, the handle 106 may be configured in any shape that would be desirable during a surgical procedure without limitation. For example, the handle 106 may be a generally ergonomically preferable shape that enables the healthcare professional to retain control over the graft-positioning system 100, but will not cause significant discomfort or pain during use of the graft-positioning system 100. In particular, in some embodiments, the cross-sectional profile of the handle 106 may include a substantially circular or otherwise curved shape, resulting in an essentially cylindrical shape. However, in other embodiments the cross-sectional profile of the handle 106 may be any other suitable shape including, but not limited to: square, rectangular, oval, regular or irregular polygonal, and any other suitable cross-sectional profile.

In various embodiments, the handle 106 may include one or more additional features to enhance the grip of the healthcare professional on the handle 106 during use. Referring to FIG. 1, the handle 106 may include one or more indentations 134A, 134B, 134C, 134D, and 134E configured to receive one or more fingers, thereby enhancing the non-slip engagement to the hand of the healthcare professional with the handle 106. In various other embodiments (not illustrated), the handle 106 may include one or more surface textures to further enhance the grip of healthcare professional on the handle 106 during use. The handle 106 may include any suitable non-slip surface texture without limitation including, but not limited to: knurling, roughening, raised ridges, surface projections, and any other known non-slip surface texture.

Referring back to FIG. 1, in some embodiments the guide rest 108 may be configured to enable the healthcare professional using the graft-positioning system 100 to at least partially engage a site within a patient. In particular, the guide rest 108 may extend from the second end 112 of the shaft 104 in a substantially perpendicular manner. As a result, in some embodiments the guide rest 108 and the handle 106 may be oriented in a generally parallel manner with respect to each other, with both the handle 106 and the guide rest 108 being substantially perpendicular to the shaft 104.

In some embodiments, at least one flange 114 may be coupled to the guide rest 108. For example, as shown in FIGS. 1-5, the flange 114 is movably coupled to the guide rest 108 such that the healthcare professional using the graft-positioning system 100 may adjust the position (i.e., the vertical position) of the flange 114. In other words, in some embodiments the flange 114 may be coupled to the guide rest 108 such that the healthcare professional employing the graft-positioning system 100 may move the flange 114 to a position more proximal to the shaft 104 or a position more distal to the shaft 104, along a vertical length of the guide rest 108. As a result, the flange 114 may be adjusted to enable the graft-positioning system 100 to engage different sized, shaped, and contoured bones within the patient.

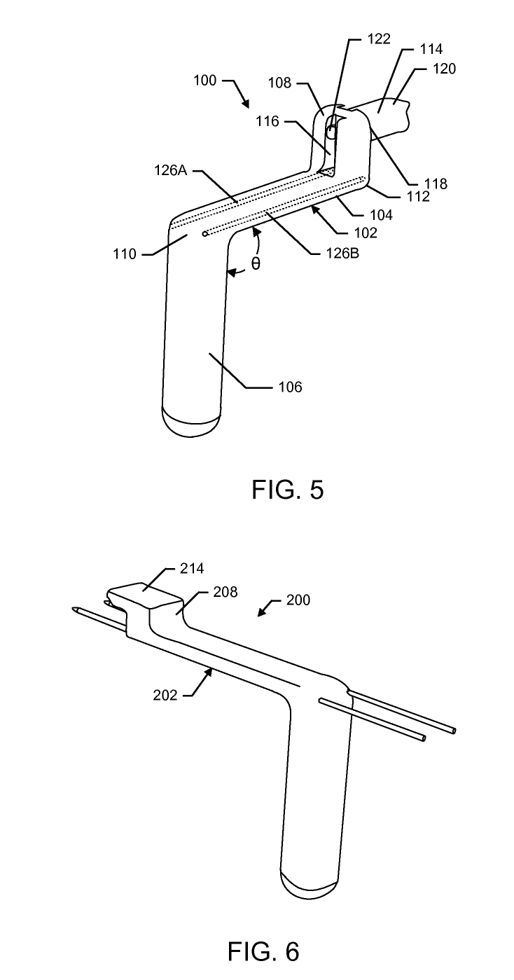

In some embodiments, the flange 114 and the guide rest 108 may be configured to be coupled to each other. As best viewed in FIGS. 4 and 5, a guide channel 116 may be defined by the guide rest 108. For example, the guide channel 116 may be dimensioned and arranged to receive at least a portion of the flange 114. In particular, in some embodiments, the flange 114 may define a coupling end 118 and a contact end 120. Moreover, the coupling end 118 of the flange 114 may be configured to be at least partially received within the guide channel 116 to provide an interface to couple together the flange 114 and the guide rest 108. For example, the coupling end 118 may be at least partially received within the guide channel 116 such that the interface may be secured, but also so that the flange 114 may move along the vertical length of the guide rest 108.

In some embodiments, a coupling device 122 may be used to provide movable coupling between the guide rest 108 and the flange 114. Specifically, the coupling end 118 of the flange 114 may define an aperture (not shown) that is configured to receive the coupling device 122. For example, the coupling device 122 may be configured as a thumb screw or as any other device that may be loosened and tightened to enable movement of the flange 114 along the vertical length of the guide rest 108. Accordingly, at least a portion of the coupling end 118 of the flange 114 may be disposed immediately adjacent to the guide channel 116 such that the coupling end 118 at least partially extends into the guide channel 116. Then, the coupling device 122 may be inserted into the aperture of the coupling end 118 through the guide channel 116. Thereafter, the healthcare professional may actuate the coupling device 122 to enable movement (e.g., vertical movement with respect to the guide rest 108) of the flange 114 with respect to the remainder of the body 102. Once the healthcare professional is satisfied with the positioning of the flange 114, the coupling device 122 may be actuated to lock the flange 114 in place.

In various embodiments, the guide channel 116, the coupling end 118 of the flange 114, and the coupling device 122 may be arranged and dimensioned to provide a controlled range of movement in a direction perpendicular to the body 104. Referring to FIG. 4, the guide channel 116 may be provided as at least one track 117A and 117B configured to receive the coupling device 122 and the coupling end 118 of the flange 114, respectively. In some embodiments, the cross-sectional profile of the coupling end 118 may be configured to translate along the track 117B with essentially no other translations or rotations in any other direction due to the close mechanical fit of the coupling end 118 within the track 117B. In other embodiments, the guide channel 116 may be provided with a slot (not shown) to provide access to the aperture of the coupling end 118 for the coupling device 122.

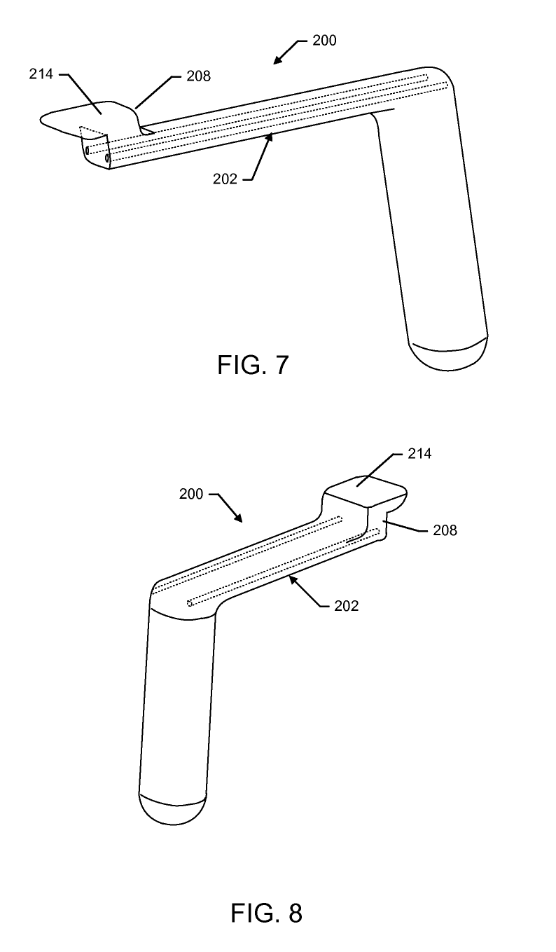

As illustrated in FIGS. 6-8, some embodiments include an alternative configuration of the graft-positioning system, designated 200. In particular, in some embodiments, the flange 214 may be immovably coupled to the guide rest 208. Specifically, the body 202 may be formed such that the flange 214 is substantially or completely integral with the guide rest 208. In other embodiments, the flange 214 may be coupled to the guide rest 208 in a manner that precludes some or all movement of the flange 214, relative to the guide rest 208. For example, the flange 214 may be welded or brazed to the guide rest 208. As a result of this configuration, the graft-positioning system 200 configured with a fixed flange 214 may be used for medical procedures where healthcare professionals are not significantly concerned about a need for adjusting the position of the flange 214. Furthermore, outside of the configuration of the fixed flange 214, the graft-positioning system 200 may generally operate in a manner substantially similar to the graft-positioning system 100. In various embodiments, two or more graft-positioning systems 200 may be provided, in which each individual graft-positioning system 200 is characterized by a different position of the flange 214 within a range of desired flange positions.

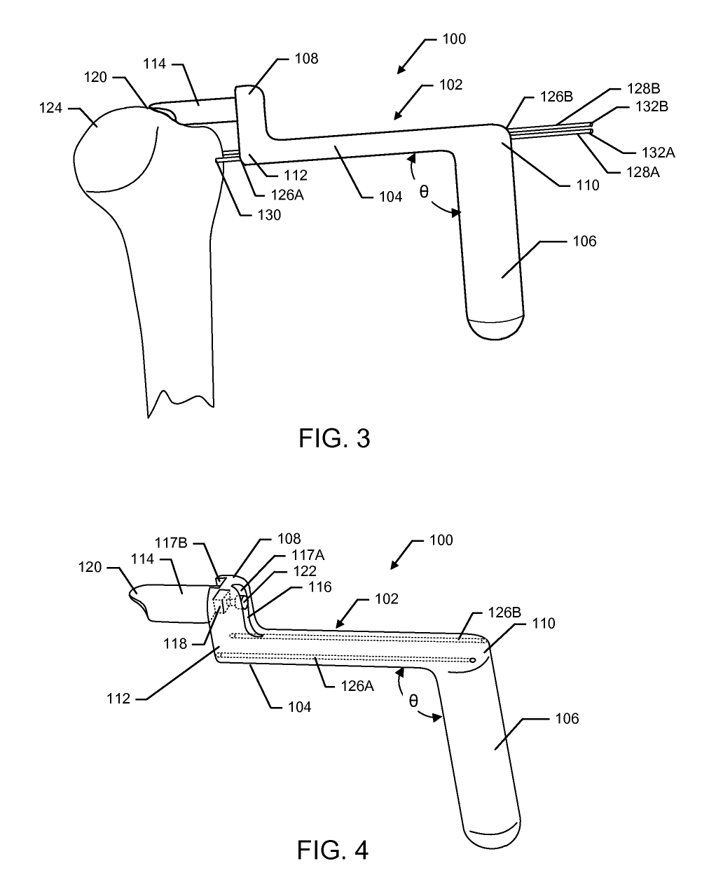

Referring back to FIG. 3, the contact end 120 of the flange 114 may be configured and arranged to engage a desired site on and/or within the patient. In some embodiments, the contact end 120 may be configured to engage a portion of a bone of the patient. In some embodiments, the contact end 120 may be configured to engage a portion of a humerus 124. For example, the contact end 120 may be configured and arranged to engage an articular surface and/or a tuberosity of the humerus 124. In particular, the contact end 120 may exhibit a generally curved, arcuate, and/or rounded-edge profile to engage the naturally non-linear/curved contours of end portions of the humerus 124. However, as previously mentioned, the graft-positioning system 100 may be used with other bones or systems in a patient without limitation. As a result, the contact end 120 may exhibit any profile that is capable of engaging a surgical site desired by the healthcare professional employing the graft-positioning system 100.

In some embodiments, the shaft 104 may define at least one pin channel 126. In particular, in some embodiments, the shaft 104 of the body 102 may define two or more pin channels 126. For example, as shown in FIGS. 4 and 5, the shaft 104 may define two pin channels 126. In one embodiment, the pin channels 126 may extend for at least a portion of a horizontal length of the shaft 104. More specifically, in one embodiment the pin channels 126 may extend from the first end 110 of the shaft 104 to the second end 112 of the shaft 104. In addition, in some embodiments the pin channels 126 may be oriented substantially parallel to each other such that the pin channels 126 extend in a substantially or completely linear path from the first end 110 to the second end 112.

In other embodiments, the pin channels 126 may be oriented in a non-parallel manner. For example, the pin channels 126 may be configured in a convergent manner. Specifically, the pin channels 126 move closer to each other (i.e., there is less lateral distance between the pin channels 126) in positions closer to the second end 112, relative to positions closer to the first end 110. Similarly, the pin channels 126 may be configured in a divergent manner. In particular, the pin channels 126 move farther apart from each other (i.e., there is greater lateral distance between the pin channels 126) in positions closer to the second end 112, relative to positions closer to the first end 110.

Referring back to FIGS. 1 and 3, one or more of the pin channels 126 may be configured to receive at least a portion of a respective guide pin 128 such that the motion of the guide pin 128 is constrained to an essentially linear path along the length of the pin channel 126. Specifically, the pin channels 126 and the corresponding guide pins 128 may exhibit a substantially similar cross-sectional profile. For example, if the guide pins 128 selected for use exhibit a substantially cylindrical configuration characterized by a circular cross-sectional profile, the pin channels 126 may exhibit a substantially similar cross-sectional profile or any other configuration that may receive the guide pins 128. As a result, the pin channels 126 may function to direct the guide pins 128 during use of the graft-positioning system 100. In some embodiments, the cross-sectional profile of each pin channel 126 may be closely matched to the cross-sectional profile of the corresponding guide pin 128 to inhibit mechanical play within the pin channel 126.

In other embodiments, the cross-sectional profile each pin channel 126 may vary in dimension and/or cross-sectional profile along at least a portion of the pin channel 126. By way of non-limiting example, the cross-sectional profile of the pin channel 126 may be relatively large near the first end 110 or the shaft 104 and may taper to a cross-sectional profile that is closely matched to the cross-sectional profile of the guide pin 128 near the second end 112 of the shaft 104. In use, this configuration may constrain the location at which the one or more guide pins 128 are inserted into the humerus 124, but may permit limited variations in the penetration angle of the guide pins 128. By way of another non-limiting example, the cross-sectional profile of a pin channel 126 may be a slot that tapers in width to permit variations in the penetration angle within a specific plane defined by the plane of the slot and within an range of penetration angles defined by the width of the slot.

In addition, in some embodiments, each of the guide pins 128 includes an engagement end 130 and a receiving end 132. In particular, during use, the guide pins 128 may be inserted through the pin channels 126 such that the engagement end 130 passes through the shaft 104 and exits the body 102 at the second end 112, while the receiving end 132 does not enter the body 102 and extends from the shaft 104 at the first end 110. In other words, in some embodiments, the guide pins 128 have a greater length than does the body 102. Moreover, in some embodiments, the guide pins 128 may be manufactured from any material suitable for use in medical procedures including, but not limited to: surgical-grade steel, aluminum, platinum, high-strength polymers, and any other suitable surgical-grade material.

In some embodiments, the guide pins 128 may be configured to engage a site on the patient. The engagement end 130 may be configured to pierce or otherwise engage a portion of a bone including, but not limited to, the humerus 124. By way of non-limiting example, the engagement end 130 of the guide pins 128 may be provided with a pointed or otherwise piercing configuration. Accordingly, the healthcare professional may actuate the receiving end 132 (e.g., via a hammer or other device capable of delivering a force to the guide pins 128) to cause the engagement end 130 to enter and remain within a portion of the humerus 124 (FIG. 3). As a result, the guide pins 128 may be engaged with, and/or reversibly coupled to, the humerus 124 to function as a guide for down-stream graft attachment, as discussed in greater detail below.

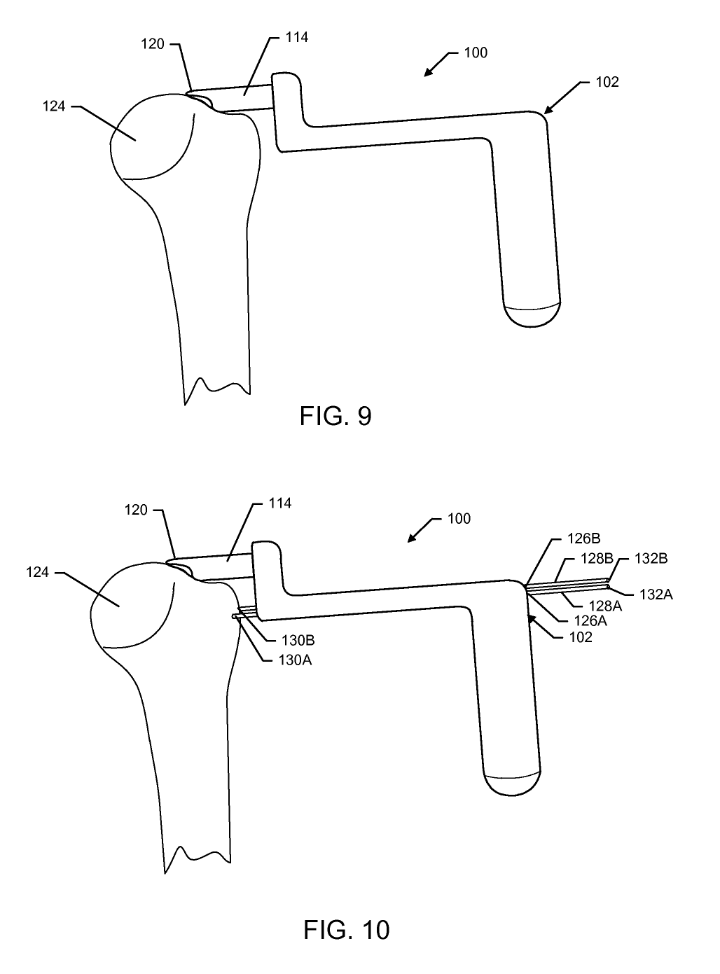

As illustrated in FIGS. 9-11, some embodiments of the graft-positioning system 100 may be used prepare the humerus 124 to receive a graft (e.g., a rotator-cuff graft). Initially, in some embodiments, depending on the size of the elements of the humerus 124 and whether the healthcare professional is employing the fixed-flange 214 embodiment (FIGS. 6-8) or the movable-flange 114 embodiment (FIGS. 1-5 and 9-11), the healthcare professional may adjust the positioning of the flange 114 to a desired location. Thereafter, as illustrated in FIG. 9, the healthcare professional may position the body 102 such that the contact end 120 of the flange 114 engages a portion of the humerus 124. For example, the contact end 120 may engage the articular surface and/or the tuberosity of the humerus 124. Moreover, in some embodiments, in addition to or in lieu of adjustment prior to engaging the flange 114 and the humerus 124, the healthcare professional may also adjust the position of the flange 114 after the contact end engages the humerus 124.

Once the healthcare professional is satisfied with the positioning of the body 102 with respect to the humerus 124, the guide pins 128 may be inserted into the pin channels 126, as shown in FIG. 10. In particular, a first guide pin 128A may be inserted into a first pin channel 126A and a second guide pin 128B may be inserted into a second pin channel 126B such that the engagement ends 130A and 130B of the guide pins 128A and 128B contact desired locations on the humerus 124. Once the guide pins 128A and 128B are positioned through the pin channels 126A and 126B and the engagement ends 130 of each of the guide pins 128 are in contact with the humerus 124, a force may be applied to the receiving ends 132A and 132B of the guide pins 128A and 128B. As a result of the force, the engagement ends 130A and 130B may be driven at least partially into the humerus 124 such that the guide pins 128 are securely engaged to the humerus 124, but removable at a later time.

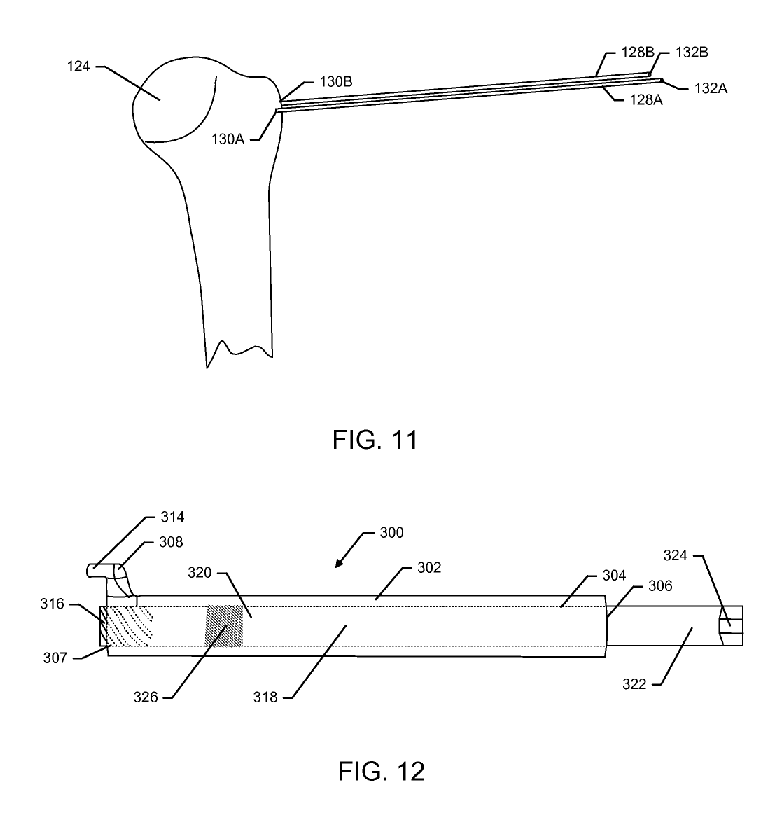

As illustrated in FIG. 11, once the guide pins 128A and 128B are securely engaged to the humerus 124, the healthcare professional may remove the body 102. For example, while holding the handle 106, the healthcare professional may pull on the body 102 in a direction opposite the humerus 124 such that the guide pins 128A and 128B remain engaged to the humerus 124 (i.e., the engagement ends 130A and 130B remain engaged to the humerus 124) and the body 102 no longer contacts any portion of the humerus 124 or the patient. If the healthcare professional is not satisfied with the positioning of the guide pins 128A and 128B, the aforementioned procedure may be repeated to ensure that the guide pins 128A and 128B are properly positioned.

If the guide pins 128 are properly positioned, the healthcare professional may use the guide pins 128 as guidance points for creating depressions for use in implanting a rotator-cuff graft (not shown). For example, once the guide pins 128 are properly positioned, a cannulated surgical drill and/or a cannulated surgical reamer may be positioned over the guide pins 128 (i.e., the surgical device includes a channel that receives the guide pins 128) to create a depression in the humerus 128 to affix the graft. In other words, the guide pins 128 serve to direct the cannulated surgical devices along an axis to ensure that the sockets are drilled at the correct angle and the correct distance apart. Thereafter, the guide pins 128 may be removed and sterilized for repeated use or discarded.

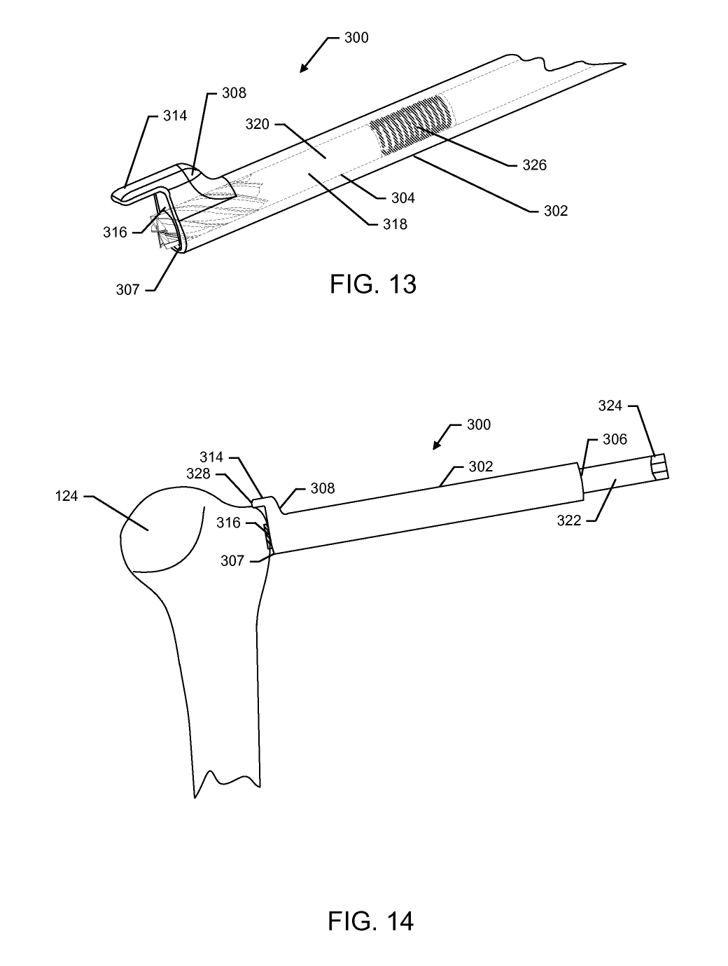

Referring to FIGS. 12 and 13, various aspects of an arthroscopic cutter are illustrated and generally indicated as 300. As described in greater detail herein, the arthroscopic cutter 300 may be employed by healthcare professionals in connection with one or more medical procedures for site preparation for implantation of various grafts. In various embodiments, the arthroscopic cutter 300 includes a tubular body 302 defining a lumen 304. The lumen 304 opens at a proximal end 306 and at an opposed distal end 307 of the tubular body 302.

In some embodiments, a guide rest 308 may be attached to the distal end 307 of the tubular body 302. The guide rest 308 may be configured to enable the healthcare professional using arthroscopic cutter 300 to at least partially engage a site within a patient in a manner similar to the guide rest 108 of the graft-positioning system 100 described herein previously. In particular, the guide rest 308 may extend from the distal end 307 of the body 302 in a substantially perpendicular manner.

In some embodiments, at least one flange 314 may form an integrated structure with the guide rest 308, thereby situating the flange 314 a fixed distance away from the body 302 in a manner similar to the fixed flange 214 of the embodiment of the graft-positioning system 200 illustrated in FIGS. 6-8 and described herein previously. In various embodiments, a variety of bodies 302 with flanges 314 situated at different heights may be used to provide the ability to vary the position of the arthroscopic cutter 300 within the surgical site. In other embodiments (not shown) the at least one flange 314 may be movably coupled to the guide rest 308 such that the healthcare professional using the graft-positioning system 100 can adjust the position (i.e., the vertical position) of the flange 314, in a manner similar to the flange 114 illustrated in FIGS. 1-5 and described herein previously.

In various embodiments, the arthroscopic cutter 300 further includes a cylindrical cutting head 316 situated within the lumen 304 near the distal end 307 of the body 302. The cutting head 316 may be attached at one end to a distal end 320 of a driveshaft 318 situated within the lumen 304. A proximal end 322 of the driveshaft 318 may protrude from the lumen 304 at the proximal end 306 of the body 302. The proximal end 322 of the driveshaft 318 may terminate in a driver fitting 324 configured to fit within a socket of a power driver capable of delivering sufficient torque to rotate the cutting head 316 at a desired rotational speed. In various aspects, the driver fitting 324 will have a cross-sectional profile matched to the cross-sectional profile of the power driver socket (not shown) including, but not limited to: a blade profile, a square profile, a hexagonal profile, an octagonal profile, and any other suitable cross-sectional profile.

In various other embodiments, the arthroscopic cutter 300 may further include a retraction spring 326 situated within the lumen 304 between the proximal end 306 and distal end 307 of the body 302. The retraction spring 326 is wound circumferentially around the drive shaft 318 and is situated between the driveshaft 318 and the inner wall of the body 302 forming the lumen 304. The retraction spring 326 is mechanically attached to the driveshaft 318 at one end and to the inner wall of the body 302 forming the lumen 304 at an opposite end. The retraction spring 326 is configured to provide a restoring force that preferentially retracts the cutting head 316 completely within the lumen 304 when no extension force is applied to the driveshaft 318.

In use, an extension force directed toward the distal end 307 of the body 302 may be applied to the driveshaft 318, causing the cutting head 316 to protrude from the lumen 304 at the distal end 307. When the extension force is removed from the driveshaft 318, the cutting head 316 passively retracts back into the lumen due to the restoring force provided by the retraction spring 326.

Some embodiments of the arthroscopic cutter 300 may be used prepare the humerus 124 to receive a graft including, but not limited to, a rotator-cuff graft. Initially, in some embodiments, depending on the size of the elements of the humerus 124 and whether the healthcare professional is employing the fixed-flange embodiment (FIGS. 12-13) or the movable-flange embodiment, the healthcare professional may adjust the positioning of the flange 114 to a desired location. Thereafter, as illustrated in FIG. 14, the healthcare professional may position the body 102 such that the contact end 328 of the flange 314 engages a portion of the humerus 124. For example, the contact end 328 may engage the articular surface and/or the tuberosity of the humerus 124. Moreover, in some embodiments, in addition to or in lieu of adjustment prior to engaging the flange 314 and the humerus 124, the healthcare professional may also adjust the position of the flange 314 after the contact end engages the humerus 124. During positioning, the cutting head 316 may be retained retracted within the lumen 304 to prevent inadvertent cutting of tissues outside of the desired surgical area.

Once the healthcare professional is satisfied with the positioning of the body 302 with respect to the humerus 124, a protrusion force may be applied to the driveshaft 318, causing the cutting head 316 to protrude from the lumen 304 and contact a surface of the humerus 124 as illustrated in FIG. 14. The driver fitting 324 may then be attached to a power driver (not shown). The power driver may be activated to rotate the cutting head 316 at a desired rotational speed. In various embodiments, the cutting head 316 may be repositioned during preparation by shifting the flange 314 along the bone surface and/or changing the vertical position of the flange 314 relative to the body 302 as described herein above. Upon completion of surface preparation, the cutting head 316 may be retracted back into the lumen 304 and the arthroscopic cutter 300 may be retracted from the surgical area. A graft-positioning system 100 may then be inserted and used to further prepare the surgical site as described herein previously.

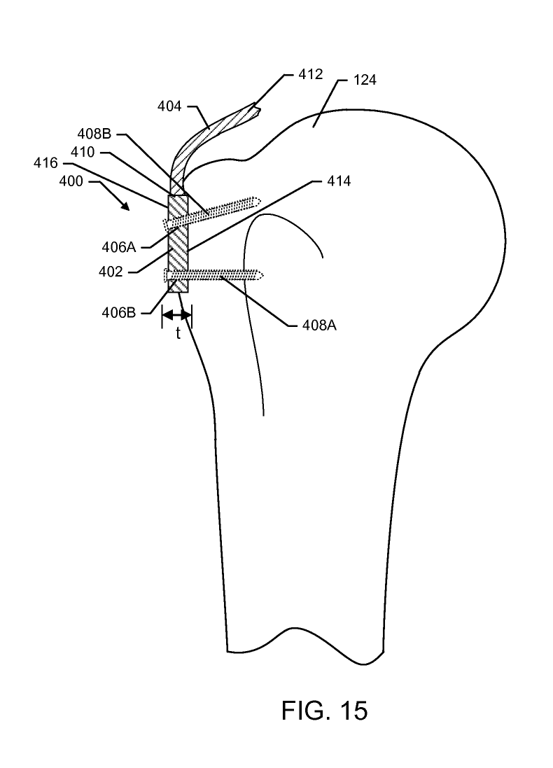

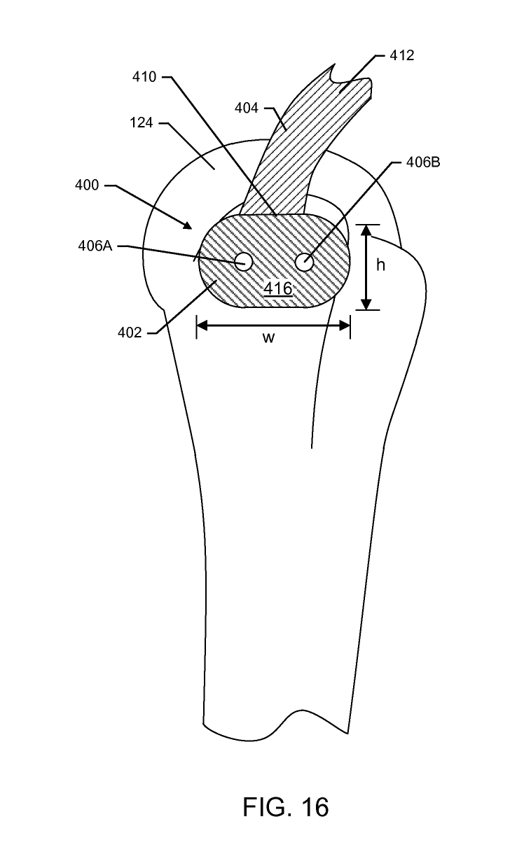

Referring to FIGS. 15 and 16, various aspects of a shell allograft are illustrated and generally indicated as 400. As described in greater detail herein, the shell allograft 400 may be implanted by healthcare professionals in connection with one or more medical procedures for the repair of rotator cuff tears. In various embodiments, the shell allograft 400 includes a bone plate 402 attached to a tendon 404. Typically, the bone plate 402 and tendon 404 are harvested as an intact unit from a donor cadaver.

In various embodiments, the shell allograft 400 may be implanted by situating a contact surface 414 of the bone plate 402 over a region of a bone including, but not limited to, a humerus 124 as illustrated in FIGS. 15-16. In some aspects, the contact surface 414 of the bone plate 402 may be contoured or otherwise shaped to conform to the external surface of the bone. In other aspects, the underlying bone surface may be contoured to conform more closely to the contour of the bone plate 402. By way of non-limiting example, the underlying bone surface may be contoured using the arthroscopic cutter 300 as described herein previously.

In some embodiments, the bone plate 402 may define at least one fastener hole 406 passing through the bone plate 402, thereby providing a path for at least one fastener 408 from the exposed surface 416 of the bone plate 402 to the contact surface 414 and into the underlying bone. The at least one fastener hole 406 may be situated at any suitable position on the bone plate without limitation. In various aspects, the bone plate 402 may define one, two, three, four, five, six, seven, eight, nine, ten, eleven, twelve, or more fastener holes 406 as needed.

In various embodiments, the tendon 404 may be attached to the bone plate 402 at a bone-tendon junction 410. In some embodiments, the shell allograft 400 may include tissues harvested from various regions of human cadavers including, but not limited to rotator cuffs and shoulder girdles. During harvest from a cadaver, the tendon, the attachment of the tendon, and the bone block underlying the attachment of the tendon may be harvested as a single intact unit. As a result, the tissue composition and the biomechanical properties of the biomaterial are substantially similar to the composition and biomechanical properties of the injured rotator cuff tissues to be replaced.

In some embodiments, the tendon 404 may terminate in a free end 412 opposite to the bone-tendon junction 410. This free end 412 of the shell allograft 400 may be cut to an appropriate size depending on any one of several factors including but not limited to: the individual morphology of the recipient patient, the extent of the rotator cuff injury or other injury to be repaired using the shell allograft 400, and/or the desired graft tendon splice type. Non-limiting examples of suitable splices of the tendon end of the biomaterial include tendon-tendon grafts and tendon-muscle grafts. The free end 412 may be joined to the native tendon or native muscle tissue using any method known in the art including absorbable sutures, non-absorbable sutures, and surgical staples. Sutures may be used to join the free end 412 to the native muscle or native tendon in any suitable suture pattern known in the art. Non-limiting examples of suitable suture patterns include a vertical mattress pattern, a horizontal mattress pattern, a crossed mattress pattern, a single running pattern, an interrupted running pattern, a running locked suture pattern, or a pulley suture pattern. The particular type of splice and method of joining may be selected based on at least one of several factors including but not limited to the location of the rotator cuff injury or other injury, the extent of the injury, the desired strength or stiffness of the join, and the desired pattern or rate of healing of the graft using the shell allograft 400.

The tendon 404 of the shell allograft may be oriented in any direction as needed to effectuate the repair of the tendon tear without limitation. In various embodiments, the tendon 404 may be aligned in proximal direction, a distal direction, a lateral direction, an axial direction, an anterior direction, a posterior direction, a cranial direction, a caudal direction, and any combination thereof. In one non-limiting example, the tendon 404 may be aligned in a proximal/cranial direction, as illustrated in FIGS. 15-16. In various embodiments, the tendon 404 may be aligned in proximal direction, a distal direction, a lateral direction, an axial direction, an anterior direction, a posterior direction, a cranial direction, a caudal direction, and any combination thereof.

The dimensions of the bone plate 402 may be any suitable dimension to provide sufficient material to secure the shell allograft 400 to the underlying bone 400 without limitation. In addition, the dimensions of the bone plate may result in suitable contact area to provide sufficient strength to the bone-tendon junction 410.

In various embodiments, the thickness t of the bone plate 402 may range from about 1 mm to about 10 mm. In other embodiments, the thickness t of the bone plate 402 may range from about 2 mm to about 6 mm. In various other embodiments, the thickness t of the bone plate 402 may range from about 1 mm to about 3 mm, from about 2 mm to about 4 mm, from about 3 mm to about 5 mm, from about 4 mm to about 6 mm, from about 5 mm to about 7 mm, from about 6 mm to about 8 mm, from about 7 mm to about 9 mm, and from about 8 mm to about 10 mm.

In various embodiments, the height h of the bone plate 402 may range from about 5 mm to about 50 mm. In other embodiments, the height h of the bone plate 402 may range from about 10 mm to about 25 mm. In various other embodiments, the height h of the bone plate 402 may range from about 5 mm to about 15 mm, from about 10 mm to about 20 mm, from about 15 mm to about 25 mm, from about 20 mm to about 30 mm, from about 25 mm to about 35 mm, from about 30 mm to about 40 mm, from about 35 mm to about 45 mm, and from about 40 mm to about 50 mm.

In various embodiments, the width w of the bone plate 402 may range from about 5 mm to about 70 mm. In other embodiments, the width w of the bone plate 402 may range from about 10 mm to about 50 mm. In various other embodiments, the width w of the bone plate 402 may range from about 5 mm to about 15 mm, from about 10 mm to about 20 mm, from about 15 mm to about 25 mm, from about 20 mm to about 30 mm, from about 25 mm to about 35 mm, from about 30 mm to about 40 mm, from about 35 mm to about 45 mm, from about 40 mm to about 50 mm, from about 45 mm to about 55 mm, from about 50 mm to about 60 mm, from about 55 mm to about 65 mm, and from about 60 mm to about 70 mm.

The planform profile of the bone plate 402, defined herein as the profile as viewed from above the exposed surface 416 as illustrated in FIG. 16, may be any shape without limitation. In various embodiments, the planform profile may be any shape that provides sufficient material to retain one or more fasteners securely to the underlying bone while blending as smoothly as possible with the outer surface of the bone. Non-limiting examples of suitable planform profiles include rectangular, regular or irregular polygonal, circular, elliptical, and any other suitable planform. In various other embodiments, the corners defining the planform may be rounded to prevent irritation or damage to the soft tissues surrounding the surgical repair.

In various embodiments, the shell allograft 400 may include bone tissue and tendon tissue from the shoulder girdle and rotator cuff. Non-limiting examples of the shell allograft 400 include a greater tuberosity bone attached to an infraspinatus tendon, a greater tuberosity bone attached to a supraspinatus tendon, a proximal humerus bone attached to a teres minor tendon, and a proximal humerus bone attached to a subscapularis tendon. In other embodiments, the shell allograft 400 may include bone tissue and tendon tissue from knee tissues and ankle tissues. Knee tissues, as defined herein, refer to bone tissues, tendon tissues, and ligament tissues of the knee joint including but not limited to patellar bone, femoral bone, tibial bone, fibular bone, patellar ligament, anterior cruciate ligament, posterior cruciate ligament, medial collateral ligament, semimembranosus tendon, and lateral collateral ligament. Ankle tissues, as defined herein, refer to bone tissues, tendon tissues, and ligament tissues of the ankle joint including but not limited to Achilles tendon, anterior inferior tibiofibular ligament, posterior inferior tibiofibular ligament, anterior talofibular ligament, posterior talofibular ligament, calcaneofibular ligament, calcaneus bone, and talus bone.

It should be understood from the foregoing that, while particular embodiments have been illustrated and described, various modifications can be made thereto without departing from the spirit and scope of the invention as will be apparent to those skilled in the art. Such changes and modifications are within the scope and teachings of this invention as defined in the claims appended hereto.

What is claimed is:

1. A graft-positioning system comprising:
a body comprising:
a shaft including a plurality of pin channels, the shaft defining a first end, and a second end, wherein the plurality of pin channels extend from the first end to the second end of the shaft,
a handle being engaged to the first end of the shaft,
a guide rest being engaged to the second end of the shaft, and
a flange being engaged to the guide rest; and
a plurality of guide pins being configured and arranged to be received within the plurality of pin channels.

2. The graft-positioning system of claim 1, wherein the handle, the guide rest, and the shaft are integral with respect to each other.

3. The graft-positioning system of claim 1, wherein the plurality of pin channels are parallel with respect to each other.

4. The graft-positioning system of claim 1, wherein the flange is movable with respect to the guide rest.

5. The graft-positioning system of claim 4, wherein the guide rest comprises a guide channel.

6. The graft-positioning system of claim 1, wherein the flange is immovable with respect to the guide rest.

7. The graft-positioning system of claim 1, wherein each of the plurality of guide pins comprises an engagement end and a receiving end.

8. The graft-positioning system of claim 1, wherein the plurality of guide pins are configured to engage a portion of a bone.

9. The graft-positioning system of claim 1, wherein the plurality of pin channels are substantially non-parallel with respect to each other.

10. A method of preparing a bone to receive a graft, the method comprising:
providing a graft-positioning system that further comprises:
a body that comprises at least one pin channel disposed therethrough and a guide rest engaged to a flange, and
at least one guide pin that is configured and arranged to be received within the at least one pin channel, wherein the at least one guide pin comprises an engagement end;
engaging the bone with the body of the graft-positioning system such that the flange contacts a portion of the bone;
positioning the at least one guide pin through the at least one pin channel such that the engagement end of the at least one guide pin contacts the bone;
actuating the at least one guide pin to engage the at least one guide pin and the bone; and
removing the body such that the at least one guide pin remains engaged to the bone.

11. The method of claim 10, comprising moving the flange with respect to the guide rest.

12. The method of claim 10, comprising forming a socket in the bone with a cannulated medical device using the at least one guide pin as a guide.

13. The method of claim 10, wherein the bone is a humerus.

14. The method of claim 10, wherein the at least one guide pin comprises a receiving end that opposes the engagement end.

15. An arthroscopic cutter comprising:
a tubular body comprising:
a lumen extending from a proximal end to a distal end of the tubular body,
a guide rest being engaged to the distal end of the tubular body, and
a flange being engaged to the guide rest; and
a drive shaft attached to a cutting head at one end and situated within the lumen; wherein,
a second end of the drive shaft opposite to the cutting head protrudes from the proximal end of the tubular body;
the cutting head is stowed within the lumen near the distal end of the tubular body in a stowed position;
the cutting head protrudes slightly from the distal end of the lumen of the tubular body in an activated position.

16. The arthroscopic cutter of claim 15, wherein the body and the guide rest are integral with respect to each other.

17. The arthroscopic cutter of claim 15, wherein the flange is movable with respect to the guide rest.

18. The arthroscopic cutter of claim 15, wherein the guide rest comprises a guide channel.

19. The arthroscopic cutter of claim 15, wherein the flange is immovable with respect to the guide rest.

20. The arthroscopic cutter of claim 15, wherein the second end of the drive shaft terminates in an attachment fitting for attaching a power driver.

21. The arthroscopic cutter of claim 15, further comprising a retraction spring wrapped circumferentially around the drive shaft between the cutting head and the second end, wherein the retraction spring is attached to the drive shaft at a first end and the retraction spring is attached to a lumen wall within the body at a second end opposite to the first end.

22. The arthroscopic cutter of claim 21, wherein the retraction spring generates essentially no force when the cutting head is in the stowed position and the retraction spring generates a retracting force when the cutting head is in the activated position.

23. A shell allograft comprising:
a bone plate comprising a contact surface and an exposed surface separated by a thickness; and
a tendon joined to the bone plate in an intact bone-tendon junction;
wherein:
the contact surface is contoured to conform with an exposed surface of a bone; and
the bone plate defines at least one fastener opening extending through the thickness of the bone plate;

24. The shell allograft of claim 23, wherein the thickness of the bone plate ranges from about 2 mm to about 6 mm.

25. The shell allograft of claim 24, wherein the height of the bone plate ranges from about 10 mm to about 25 mm.

26. The shell allograft of claim 25, wherein the width of the bone plate ranges from about 10 mm to about 50 mm.
